(19) United States
(12) Patent Application Publication
(10) Pub. No.: US 2006/0188986 A1
(43) Pub. Date: Aug. 24, 2006

(54) Restoration of methylation states in cells

(76) Inventors: Douglas Spencer Millar, New South Wales (AU); John Robert Melki, New South Wales (AU); Geoffrey Walter Chigg, New South Wales (AU); George L Gabor Miklos, New South Wales (AU)

Correspondence Address: MCDERMOTT, WILL & EMERY, 4370 LA JOLLA VILLAGE DRIVE, SUITE 700, SAN DIEGO, CA, 92122, US

(21) Appl. No.: 10/536,633

(22) PCT Filed: Nov. 26, 2003
(86) PCT No.: PCT/AU03/01573

§ 371 Date: Mar. 17, 2006

(30) Foreign Application Priority Data
Nov. 27, 2002 (AU) 2002952944

(51) Int. Cl. C12N 5/08; C12N 15/87

(52) U.S. Cl. 435/455; 435/366; 435/325; 435/372

(57) ABSTRACT

A method for altering a characteristic or state of a cell, or reprogramming a cell, comprising treating a first cell type with an agent capable of altering a characteristic or state in a cell or reprogramming a cell, and determining the degree of alteration of the treated cell by measuring the methylation signature within the genome of the treated cell, wherein a given methylation signature is indicative of an altered characteristic or state of the treated cell. The preferred substance for treating the first cell is a cellular extract, lysate or component from a second cell type, the second cell type having a desired characteristic, or being the desired cell type the first cell type is to be reprogrammed to. The examples show the methylation state of a fibroblast cell being reprogrammed to that of an immune system T cell.

### TECHNICAL FIELD

The invention relates generally to methods to alter cell characteristics and monitoring alteration by evaluating DNA methylation signatures.

### BACKGROUND ART

The complete information necessary to encode the structure of all gene products of an organism such as an animal (or a plant) is stored in the sequence of the four deoxynucleotide adenine (A), guanine (G), thymine (T) or cytosine (C) in its deoxyribonucleic acid (DNA). There is, however, a fifth deoxynucleotide in DNA produced as a result of the post-replication methylation of some of the C deoxynucleotides (mC), (Millar, D S., Holliday, R., and Grigg, W. 2003, in; The Epigenome, eds, Bedk, S and Olek, A, WILEY-VCH Veriag GmbH & Co Weinheim). One of the functions of the mC is to act as a developmental signal determining whether or not a particular gene is active and able to be transcribed in order for its gene product to be made, (Li E., 1999, Nature Genetics, 23, 5-7; Coffigny H., et al., 1999, Cytogenetics and Cell Genetics, 87, 175-181). In general, the methylated state signals silencing of a gene, (Lunyak, V V., 2002, Science, 298, 1747-1752) and the unmethylated state signals activation of a gene of cells of different tissue types, (Moreau P., et al., 2003, Proc. Natl. Acad. Sci. USA., 100, 1191-1196). Methylation can change in a coordinated fashion according to a genetically controlled pattern at various stages in the development of a whole adult from a fertilised egg, (Monk M., 1995, Dev Genet., 17, 188-197). The precise way in which this happens, its causes and how these processes are controlled are yet to be discovered.

Methylation signatures in differentiated adult cells of different cell types differ from each other. Normally these different signatures of methylation are quite stable through many cell divisions but under certain. circumstances they can be modified. For example, DNA methylation is thought to be part of the process involved in the cloning of animals, where the nucleus from an adult fully differentiated cell (such as an epithelial cell) is inserted into the cytoplasm of an enucleated embryonic stem cell. The epithelial cell nucleus is reprogrammed so that it takes on the developmental potential of the embryonic stem cell cytoplasm. This process is thought to involve the reprogramming of the DNA methylation signature of the genome of the epithelial nucleus. It is generally thought that incomplete or abnormal epigenetic reprogramming of the epithelial nucleus is the basis for the low success rate of cloning, (Pennisi E, 2001, Science, 293, 1-064-1067; Dean W et al., 2001, Proc. Natl. Acad. Sci. USA, 98,13734-13738; Bourc'his, D et al., Current Biology, 11, 1542-1546; Humphreys D et al., 2001, Science, 293, 95-97; Kang, Y-K.,: 2001, Nature Genetics, 28, 173-177; Mann M R W and Bartolomei, M S., 2002, GenomeBiology, 3, 1003.1-1003.4)

Modification of the normal methylation signature in the genome is often deleterious to the health of the individual, and in humans, can lead to life-threatening diseases such as cancer, (Jones, P A., 1996, Cancer Research, 56, 2463-2467; Paz et al., 2003, Human Molecular Genetics, 12, 2209-2219; Baylin et al., 2001, Human Molecular Genetics, 10, 687-692; Wei et al., 2003, Ann N Y Acad Sciences 983, 243-250; Toyota et al., 2003, Proc. Natl. Acad Sci. USA, 100, 7818-7823), and to a diversity of other problems such as neurological disorders, fragile X syndromes and so forth, (Kriaucionis and Bird, 2003, Human Molecular Genetics, 12, R221-R227; Robertson, K D and Wolffe, A p, 2000, Nature Reviews Genetics, 1, 11-19; Esteller M., et al., 2002 Clinical Immunology, 103, 213-216; Feinberg, A P et al., 2002, Cancer Research, 62, 6784-6787); This has lead to the clinical usage of some methods which are known to modify methylation of cells in tissue culture, such as treating the cells with 5-azacytidine or 5 azadeoxycytidine which inhibits the function of the methylating enzyme 5-methyl transferase and after further rounds, of DNA replication, leads to global de-methylation of many genes in the genome, (Pietrobono R et al., 2002, Nucleic Acids Research, 30, 3278-3285). Whilst it has had limited success in treating certain types of cancer, it also has toxic side effects.

So far it has not proven possible to selectively and co-ordinately de-methylate or methylate specific Cs in the genome of living cells. The goal of reprogramming methylation states of aged or diseased cells in a coordinated fashion in order to restore the normal or younger methylation signatures to date has only been a dream.

As individual cells age or are exposed,to environmental perturbations of various types their genomes may be damaged, (Richardson B, 2003, Ageing Research Reviews, 2, 245-261; Nakajima, T., et al., 2001, Int J Cancer, 94, 20821 1; Issa, J P 2003, in; The Epigenome, eds, Beck, S and Olek, A, WILEY-VCH Verlag GmbH & Co Weinheim). Normally, such damage is repaired. However, errors occur in the repair processes which lead to changes in the genetic code in the cell's DNA and thus to mutations if the modification is limited to a change in one or other of the four coding nucleotides A, G, T or C. If, however, the modification involves a change in the signature of 5′ methyl cytosine (5 mC) nucleotides in the controlling or regulatory regions of genes, it can result-in gene expression being activated if a particular 5 mC is replaced by a C, or gene expression being silenced if a C is replaced by a 5 mC. DNA-damaging agents such as certain drugs or ionising radiation can produce such a modification of the methyl cytosine (mC) signature in genomes, (Nyce, J W., 1997, Mutation Research, 386, 153-161). Moreover, with the passage of time, cells accumulate such methylation/demethylation changes in their DNA which have the effect of modifying in a deleterious fashion normal cellular function. This can result in increasing levels of disability of the aging individual or may predispose to a disease such as various types of cancer in the individual having such damaged cells.

The present inventors have devised a means of global but specific directional reprogramming of the mC signature in cells in order to overcome or alter the deleterious effects of accumulated abnormal methylation changes in the mC signature in cells.

### DISCLOSURE OF INVENTION

In a first aspect, the present invention provides a method for altering a characteristic or state of a cell comprising:

treating a first cell type with an agent capable of altering a characteristic or state in a cell; and

determining the degree of alteration in the treated cell type by measuring a DNA methylation signature within the genome of the treated cell type, wherein a given DNA methylation signature is indicative of an altered characteristic or state of the treated cell type.

In a second aspect, the present invention provides a method for altering a characteristic or state of a cell comprising:

treating a first cell type having an undesired characteristic or state with an extract, lysate or cellular component from a second cell type having a desired characteristic or state under suitable conditions and period of time to alter a characteristic or state of the first cell type; and

determining the degree of alteration of the treated cell type by measuring a DNA methylation signature within the genome of the cell, wherein a given methylation signature is indicative of a desired characteristic or state in the cell.

The method may further include:

pre-treating the first cell type so as to make the cell permeable to macromolecules:

The method may also further include:

culturing or growing the treated cell to obtain multiple copies of the treated cell.

The first cell type may be any existing cell type of the human hematopoietic lineage, (including cells from birth onwards to about 48 hours post mortem, as well as cells derived from the umbilical cord, the placenta, or cells from cell lines that are derivatives of the above cell types) or any other cell that is taken from elsewhere in the human body and which is reprogrammed into the hematopoietic lineage.

The term DNA methylation signature within the genome of the cell is defined as a group of cytosine within a region of the human genome that has a characteristic methylation signature which corresponds to a specific cell type. The signature can be determined in any given cell type or cell sub-type by determining the specific methylation profiles or patterns of one or more cystosines associated with one or more areas of genomic DNA. For example, the signature may be a cystosine methylation pattern associated with one or coding regions in the DNA. Such a signature will be diagnostic for example for a CD14+ monocyte, or for a CD34+ stem cell, or for a trajectory within a cell type such as an old or younger stem cell. This signature of modified, cytosines is an indicator of that cell type without recourse to cell type characteristics such as cell surface molecules, combinations of proteins within a cell, mRNA expression, metabolite concentrations, cellular inclusions, morphological characteristics determined at various microscopic levels, (light or electron microscope), or combinations of the above.

Preferably, the first cell type is a cell derived from an individual suffering from age-related disabilities, or from a disease such as cancer, or from an autoimmune disease, (van Laar, J M and Tyndall, R, 2003, Cancer Control, 10, 57-), or from cardiovascular problems such as myocardial infarction or ischemia, (Perin E C et al., 2003, Circulation, 107, 2294-23802). More preferably, the first cell type is a stem cell. It will be appreciated, however, that other cell types, such as T cells or monocytes of the immune and hematopoietic system, (Abbas A K., 2000, Cellular and Molecular Immunology, 4^{th} Edition, W B Saunders and Company; von Adrian U H., et al., 2000, New Engl. J Med, 343, 1020-1034), particularly diseased cell types could be treated.

The agent may be a chemical drug, nucleic acid, aptamer, antibody, antigen, intercalating nucleic acid (INA), peptide nucleic acid. (PNA), Locked Nucleic Acid (LNA), Hexitol Nucleic Acid (HNA), Altritol Nucleic Acid (ANA), Cyclohexanyl Nucleic Acid (CNA), oligonucleotide, modified oligonucleotide, single stranded DNA, RNA, protein, peptide, a combination thereof, or chimeric versions thereof.

In a preferred form, the agent is an extract, lysate or cellular component from a second cell type having a desired characteristic or state.

The second cell type can be derived from any species or combination of species in which the DNA methylation profile of the cell is the desired end point for reprogramming of first cell type. Preferably, the second cell type is a cell derived from a normal or healthy individual of a cell type similar to the first cell type. More preferably, the second cell type is a stem cell, (Orkin, S H., Zon, L I., 2002, Nature Immunology, 3, 323-328; Gage, F H., and Verma, I M., 2003, Proc. Natl. Acad. Sci. USA, 100, 11817-11818; Prockop D J., et al., 2003, Proc Natl. Acad. Sci. USA, 100, 11917-11923). The second cell type can be from a sub adult or an individual not suffering from a condition or state caused by an undesired characteristic of the first cell type. It will be appreciated that the other cell types such as bone marrow stem cells, other stem cells, and epithelial cells could also be used as the second cell type.

In one preferred form, the first cell type and the second cell type are of the same cell type from the same species. In another preferred form, the first cell type and the second cell type are not of the same cell type or not of the same species. Examples of second cell types include, but not limited to amphibian egg cells or germline cells for use on human or other mammalian first type cells.

By stem cells, it is meant to include all adult stem cells and particularly those of bone marrow lineages, (Verfaillie, C M., 2002, Nature Immunology, 3, 314-317; Orkin, S H., Zon, L I., 2002, Nature Immunology, 3, 323-328; Gage, F H., and Verma, I M., 2003, Proc. Natl. Acad. Sci. USA, 100, 11817-11818; Prockop D J., et al., 2003, Proc Natl. Acad. Sci. USA, 100, 11917-11923).

If required, the first cell type can be treated by any suitable means to render it permeable to the passage of macromolecules. Treatment includes, but is not limited to, electroporation, low temperature thermal shock, or various enzymes such as streptolysin O. More preferably, the treatment renders the cell temporally permeable.

The extract, lysate or cellular component can be obtained by any suitable means known to the art. Examples include, but not limited to, those described by Hakelien et al (2002) Nature Biotechnology 20:460-466. It will be appreciated that the cell-free extract may be further processed or fractionated to obtain components or macromolecules from the second cell type that, when in a first cell type, will alter the DNA methylation signature within the genome of the first cell type.

The exposure time can be from minutes to hours, depending on the cell type, extract, lysate or cellular component and the conditions of treatment. The treatment may be done at physiological temperature, or any other temperature which will not result in the death of the cell.

The treated or reprogrammed cell may be cultured in any suitable media or host known to the art under conditions that are, suitable for cell growth and division. The present invention allows the production or selection of stable treated or reprogrammed cells having a desired characteristic confirmed by its methylation signature in its genomic DNA.

The host may be an animal, either vertebrate or invertebrate. When the cells have been suitably cultured in the host, they may be removed from the host by any means known, so as to leave the cells intact. In one preferred form, the host is a domestic animal selected from bovine, ovine, equine, poultry, or porcine.

Preferably, the methylation signature within the genome of the cell is determined by diffusible factors present within the host.

The methylation signature is preferably determined by bisulphite treatment assays known to the art or developed by the present applicant.

In a preferred form, for example the reprogramming of aged cells to a younger state, confirmation of the successful treatment is the DNA methylation signature being restored to the “normal” younger signature in at least part or the whole of the genome. It will also be appreciated that methylation of only a part of the genome DNA can also occur.

The method of the invention can be used to provide a desired characteristic or state in a first cell type such that DNA methylation signature characteristic of one cell subtype may be changed to that characteristic of another subtype. For example, from one lymphocyte T cell subtype to another lymphocyte T cell subtype, or an old heratopoietic stem cell may be modified to a younger hematopoietic stem cell.

Alternatively, the method of the invention can be used to provide a desired characteristic or state in a selected population of cells within a mixed population. For example, if there is a mixed population of cells, say cancerous and normal, the mixed population of cells may be treated or targeted by the method of the invention such that only the normal cells are altered, respond and divide. By determining the DNA methylation signature of the treated cells, it can be confirmed that the normal cells have been suitably altered to have the desired effect. Whilst not changing cancer cells to normal, the method can be used to increase the proportion of one desired cell type over another.

In addition, in a mixed population of cells, such as normal and cancerous, the cancer cells can be treated or reprogrammed to enter a dormant or apoptotic, or suicide state. In the general case of two cell types, or two cell subtypes, or multiple cell types or multiple cell subtypes, any type or subtype can be treated or reprogrammed to provide a competitive advantage over any other cell type.

The method may result in the reprogramming of a modified methylation signature of a single cell type to that of the signature of a corresponding normal cell of the same cell type, (intra cell type reprogramming).

The method may be used to reprogram the methylation signature of old cells to that of younger cells, (Geiger, H and Zant, G V., 2002, Nature Immunology, 3, 329-333). For example, the methylation signature of old adult stem cells can be reprogrammed to a signature of younger stem cells. The methylation signature of the genome of diseased stem cells can be altered to that of normal stem cells, and the methylation signature of the genome of other diseased cells, such as T cells of the immune system, can be altered to that of non diseased T cells.

The stem cells can be those cells whose DNA has been corrupted by exposure to drugs, such as chemotherapeutic drugs, or common medications, or damaging electromagnetic radiation, or inadequate supplies of normal chemicals such as folate during pregnancy, for example, (Waterlahd R A and Jirtle, R L., 2003, Molecular and Cellular Biology, 23, 5293-5300; Fenech M., 2003; in; The Epigenome, eds, Beck, S and Olek, A, WILEY-VCH Verlag GmbH & Co Weinheim).

In a third aspect, the present invention provides an isolated altered or reprogrammed cell obtained from the method according to the first or secbnd aspects of the present invention.

Preferably, the treated or reprogrammed cell is a stem cell: The treated or reprogrammed cells can be used as the second cell type for subsequent treatments which would obviate the need to obtain fresh second cell types from individuals each time a treatment is carried out. The treated or reprogrammed cells may also be useful as cells lines for research or other medical uses such as cell therapy or transplantation.

In a fourth aspect, the present invention provides a method for treating an individual suffering from a condition resulting from having cells with an undesired characteristic or state, the method comprising:

obtaining cells from the individual;

carrying out the method according to the first or second aspects of the present invention on at least some of the cells to obtain treated or reprogrammed cells having a desired characteristic or state; and

returning the treated or reprogrammed cells to the individual where the cells multiply and replace at least some cells having the undesired characteristic so as to treat the condition.

The ctells may be a heterologous cell population or a given cell type. The cells may be of an undesired type such as cancerous or a desired type such as normal cells.

Preferably, the individual is suffering from a condition such as disabilities associated with aging or with cancer or with autoimmune disease.

In a preferred form, the cells are stem cells. The treated or reprogrammed stem cells can then differentiate in the subject to assist in the treatment. It will be appreciated that the other cell types such as cancer cells or those associated with other diseases could also be treated.

The present invention is particularly useful to restore characteristics in dysfunctional cells havingr undesired characteristic(s) or state(s) due to DNA methylation signatures which have been modified as a consequence of age-related processes, by exposure to drugs, or to more random events.

An advantage of the present invention is that an individual's cells, after treatment or reprogramming are returned (transplanted) to the individual. Accordingly, there should not be any problems of rejection or the requirement to use immunosuppressive drugs or medication.

The method can be used for individual or personalised treatment depending on the subject and condition.

Cells which have been made temporarily permeable to macromolecules can be treated with a cell-free extract, lysate, or ceIlular component of normal cells of the same phenotype. Such cells may be derived from the treated host individual or more preferably from extract, lysate or cellular component extracted or obtained from cells derived from a younger individual who has not been exposed to the same environmental perturbations (such as age) which caused the problem.

The present invention may also be employed where extracts, lystaes or cellular components of a second cell type are used only as a way of getting a particular combination of molecules that may send human cells from one cell type to another by novel routes. In summary, cancerous cells are highly aneuploid and have grossly rearranged genomes, hence some genes are highly amplified. In addition, cancerous cells do not correspond to any known cell type as each cancer cell is probably unique. Hence they may provide a source of extract from which one could molecularly extract, say, high levels of a particular protein. In addition, because cancer cells often contain chromosomal translocations, they often have fused genes which produce novel protein products. These novel protein products could be used to treat or reprogram cells in a better way than using extract from existing conventional cell types.

Similarly, many organisms have rearranged genomes that could be used for these purposes of getting novel proteins or RNAs from their cell populations. Thus strains of mice, mutant mice, transgenic mice, mice infected with viruses, will all have cell populations that are uniquely suitable for extracts.

Similarly the huge range of Drosophila mutants, C. elegans mutants, yeast mutants, E. coli mutants etc and their transgenic derivatives could all provide novel gene products in extracts, fused genes, gain of function mutations such as neomorphs, novel forms of small RNAs etc. The availability of such novel proteins and RNAs from not only the natural evolutionary spectrum, but from the huge warehouses of mutant strains, can be used by the present invention.

In a fifth aspect, the present invention provides use of an isolated altered cell according to the third aspect of the present invention in a method of therapy.

Preferably, the therapy is cell therapy.

In a sixth aspect, the present invention provides use of an isolated altered cell according to the third aspect of the present invention in the manufacture of a medicament for therapy.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 shows an agarose gel electrophoresis separation of total RNA isolated from adult peripheral blood stem cells in order to determine that the RNA was of high quality prior to analysis on Affymetrix microarray platforms. Lane M shows the molecular weight markers. Lane SE488 and Lane SE489 shows RNA from two individuals. The two prominent bands represent the 18S and 28S ribosomal RNA.

FIG. 2 shows an Agarose gel electrophoresis separation of the RNeasy purified sample to determine the quality of the RNA prior to further processing. Lane M shows the molecular weight markers. Lane SE488 shows the RNA from a pool from both individuals. The two prominent bands represent the 18S and 28S ribosomal RNA. Results are consistent with high quality RNA.

FIG. 3 shows an agarose gel electrophoresis separation of purified cRNA. Lane M shows the molecular weight markers. Lane SE488 shows the cRNA from a pool from both individuals (SE488).

FIG. 4 shows verification of cRNA fragmentation by agarose gel, electrophoresis. Lane M shows the molecular weight markers. Lane SE488 shows the fragmented cRNA from a pool from both individuals.

FIG. 5 shows microarray analysis of the expression profiles of young stem cells versus old stem cells using the Atlas 8K microarray. Each probe on the array is for hybridization to a transcript from a human gene and each probe was spotted twice, hence the doublet spots. Radioactively-labelled RNA from a cell population was hybridized to the array and the signal strength of the doublet spots indicates the expression level of the particular gene in this particular cell type.

FIG. 6 shows DNA sequences from regions of the human genome harbouring seven selected loci termed ABCB1, IRF7, ESR1B, GZMA, CDX1, MAGEA2 and THY1 and their methylation status as determined by DNA sequencing after bisulphite modification of the DNA extracted from cells before reprogramming and after reprogramming. First cell type (the cell to be treated or reprogrammed) represents the cell type prior to any treatment. Second cell type represents the desired endpoint towards which the first cell type needs to be shifted. “Reprog”, denotes the methylation status of genomic DNA sequences from cells that have been treated with extract from the second cell type. The changes at the DNA level are a result of the treatment.

### MODE(S) FOR CARRYING OUT THE INVENTION

Methods

Determining Methylation Signatures of Cells

Bisulphite Treatment of DNA

To 2 μg of DNA, which can be pre-digested with suitable restriction enzymes if so desired, 2 μl ( 1/10 volume) of 3 M NaOH (6 g in 50 ml water, freshly made) was added in a final volume of 20 μl. The mixture was incubated at 37° C. for 15 minutes. Incubation at temperatures above room temperature can be used to improve the efficiency of denaturation.

After the incubation, 208 μl 2M Sodium Metabisulphite (7.6 g in 20 ml water with 416 ml 10 N NaOH; BDH AnalaR #10356.4D; freshly made) and 12 μl of 10 mM Quinol (0.055 g in 50 ml water, BDH AnaIR #103122E; freshly made) were added in succession. The sample was overlaid with 200 μl of mineral oil. The sample was then incubated overnight at 55° C. Alternatively the samples can be cycled in a thermal cycler as follows: incubate for about 4 hours or overnight as follows: Step 1, 55° C./2 hr cycled in PGCR machine; Step 2, 95° C./2 min. Step 1 can be performed at any temperature from about 37° C. to about 90° C., and can vary in length from 5 minutes to 8 hours. Step 2 can be performed at any temperature from about 70° C. to about 99° C. and can vary in length from about 1 second to 60 minutes, or longer.

After the treatment with Sodium Metabisulphite, the oil was removed, and 1 μl tRNA (20 mg/ml) or 2 [l glycogen were added if the DNA concentration was low. These additives are optional and can be used to improve the yield of DNA obtained by co-precipitating with the target DNA especially when the DNA is present at low concentrations.

An isopropanol cleanup treatment was performed as follows: 800 μl of water were added to the sample, mixed and then 1 ml isopropanol was added. The sample was mixed again and left at −20° C. for a minimum of 5 minutes. The sample was spun in a microfuge for 10-15 minutes and the pellet was washed 2× with 80% ETOH, vortexing each time. This washing treatment removes any residual salts that precipitated with the nucleic acids.

The pellet was allowed to dry and then resuspended in a suitable volume of T/E 10 (10 mM Tris/0.1 mM EDTA) pH 7.0-12.5 such as 50 μl. Buffer at pH 10.5 has been found to be particularly effective. The sample was incubated at 37° C. to 95° C. for 1 min to 96 hr, as needed to suspend the nucleic acids.

PCR amplification was performed on 1 μl of treated DNA, PCR amplifications were performed in 25 μl reaction mixtures containing 1 μl of bisulphite-treated genomic DNA, using the Promega PCR master mix, 6 ng/μl of each of the primers. One μl of 1^{st} round amplification was transferred to the second round amplification reaction mixtures containing primers. Samples of PCR products were amplified in a ThermoHybaid PX2 thermal cycler under the conditions described in Clark et al. Nucleic Acids Res. Aug. 11, 1994; 22(1 5):2990-7.

Agarose gels (2%) were prepared in 1% TAE containing 1 drop ethidium bromide (CLP #5450) per 50 ml of agarose. Five μl of the PCR derived product was mixed with 1 μl of 5× agarose loading buffer and electrophoresed at 125 mA in X1 TAE using a submarine horizontal electrophoresis tank. Markers were the low 100-1000 bp type. Gels were visualised under UV irradiation using the Kodak UVIdoc EDAS 290 system.

Treatment or Reprogramming Procedure

Second cell Type for making extract ie Jurkat T-cell line (1× Roller Bottle 1,700 cm²)
First cell Type to be treated, or reprogrammed, i.e. 293T fibroblasts cell (2×T75 flasks having been split the day prior to reprogramming).
DMEM growth media for cells
Trypsin/EDTA (0.25%)
Cell Lysis Buffer i.e. Base solution of 50 mM NaCl, 5 mM MgCl₂, 20 mM Hepes pH 8.2 can be stored frozen in 50 ml aliquots. Later additives for buffer were:
   PMSF (100 mM stock in ETOH), DTT (1 M-stock) and Protease Inhibitor Cocktail, aliquoted and frozen (Sigma P8340).
Streptolysin O (Sigma S0149) stock aliquoted and frozen at 65 units/μl in H₂O. (Frozen stocks should be discarded after 1 month).
HBSS (Hanks Balanced Salt Solution, —Ca²⁺-free)
1×500 ml bottle of-PBS (Ca²⁺/Mg²⁺-free)
200 mM CaCl₂
ATP (200 mM stock in water)
GTP (10 mM stock in water)
Phosphocreatine (2 M stock in water)
Creatine kinase (5 mg/ml stock in water)
Nucleotide (NTP) mix (ATP, GTP, CTP, UTP; 100 mM of each)
Dextran, Texas Red (Molecular Probes D-1830) 25 mg/ml in water.
Probe Sonicator with 3 mm tip
Multi-well plates for cell-culture (48 wells)
Disposable 50 ml, 15 ml, 1.5 ml and 0.2 ml tubes (sterile)
Centrifuges at 4° C.:
   centrifuge with swing-out buckets for 1.5 ml tubes
   centrifuge for 1.5 ml tubes for spinning at 15 000 g
   centrifuge for 15 ml and 50 ml tubes at 800 g.
Microscope slides and coverslips
Cell Chamber slides
Water bath at 37° C.
Standard Confocal Microscope
Epifluorescence Microscope with green fluoro filter.
   Preparation of Extract
I. Harvest second cell type Jurkat T-cell line cells and transfer to 50 ml tubes, spin 800 g (˜1,200 rpm) 10 minutes at 4° C.
II. Wash cells 2× in cold 1×PBS, spin 800 g 10 min, 4° C. Pool cells into one 15 ml tube with graduation lines during first wash. Take out a small aliquot for counting cells while doing a second wash.
III. Wash cell pellet 1× in 10 ml ice-cold cell Lysis buffer with addition of:
   1 μl 1M DTT/ml of buffer and 10 μl 100 mM PMSF/ml of buffer.
IV. Spin 800 g, 10 min., 4° C. Important to keep cells on ice after adding Lysis buffer.
V. Estimate the volume of the cell pellet and resuspend the pellet in slightly less than 1× volume of Lysis buffer with DTT, PMSF and Protease Inhibitor Cocktail (10 μl cocktail/ml of buffer).
   *NB The cell suspension is very thick; try not to introduce air bubbles.
   Use the pipette to see if the final volume is 2× cell pellet and if not add more buffer. Do not to dilute the cell pellet any more than 2× the original pellet volume.
VI. Leave on ice for 45 min for cells to swell.
VII. Aliquot cells into 200 μl in 1.5 ml tubes. Sonicate until all cells and nuclei are lysed (check under microscope). 35% power, 0,4 s intervals, approx. 1 min 15 sec per tube. (Sonication conditions have to be optimized for each cell type.)
VIII. Pool lysates into 1.5 ml tubes, spin 15 000 g, 15 min at 4° C.
IX. Take off the supernatant (extract) and keep aside 3×20 μl aliquots of extract for testing protein concentration, pH and toxicity. Aliquot the remainder into 200 μl-PCR tubes with 100 μl extract/tube.
X. Use the extract directly or snap freeze in liquid N₂ or dryice and ETOH and store at −80° C.
   Cell Treatment or Reprogramming
I. Harvest target 293T fibroblasts cells (first cell type) and wash 2× in cold 1×PBS. Keep cells cold during washing. Take out an aliquot for counting during second wash. Determine number of cells.
II. Wash cells 1× in cold HBSS.
III. Resuspend cells to give a concentration for each reaction of 20,000 cells/100 μl HBSS in 1.5 ml tubes. As cell numbers are low in each reaction set up 5× each reaction so when combined into final plate wells there will be 100,000 cells per well. This is also for controls.
IV. Spin in swing-out bucket rotor, 5 min, 1200 rpm, 4° C.
V. Remove supernatant (be careful not to remove cells)
VI. Add 15.5 μl HBSS to each tube.
VII. Prepare SLO working solution: Thaw aliquot of stock (65.8 units/μl), keep on ice. Dilute 1:100 in ice-cold HBSS. Keep on ice at all times
VIII. Place tubes in water bath, 37° C. Preheat samples for a couple of minutes before adding SLO.
IX. Add 4:5 μl SLO/tube (1:100 dilution, gives 2.9 units final SLO concentration), gently flick tubes to mix SLO with cell suspension.
X. Incubate for 50 minutes at 37° C.,
XI. Prepare extract for reprogramming:
   →Prepare ATP generating system by mixing the ATP, GTP, creatine kinase, phosphocreatine stocks at 1:1:1:1 ratio (stocks can be kept at −20° C.).
   100 mM NTPs are mixed 1:1:1:1 to give a 25 mM stock which can be frozen, once thawed keep on ice.
XII. To each 100 μl of extract needed, add 5 μl of above freshly mixed ATP generating system and 4 μl of NTP mix. Keep extract on ice until use.
XIII. Put tubes (with cells in SLO) on ice and add 200 μl ice-cold HBSS to each tube (to stop SLO reaction). Spin 5 min, 4° C., 1200 rpm in swing-out bucket rotor. Put tubes back on ice.
XIV. Remove supernatant. Be careful not to remove cells.
XV. Add 20 μl extract cocktail/tube of cells(or 20 μl HBSS with Texas Red dextran for uptake control)
XVI. Flick tubes gently to mix. Incubate 1 h at 37° C. (water bath). Transfer tubes to rack (room temp) and add culture medium with 2 mM CaCl₂.
XVII. Transfer cells to multi-well cell culture plate pooling the 5 reactions into one well.
XVIII. Incubate cells 2 hours in CO₂ incubator.
XIX. Replace CaCl₂ medium with regular culture medium and leave overnight.
   Expression Profiling of Aged Individuals
   Microarray Profiling Procedures; Expression Profiling of Stem Cells from Young and Aged Individuals

Adult CD34+ sterh cells were mobilized into the peripheral bloodstream using standard G-CSF protocols (Filgrastim; de al Rubia et al., 2002, Transfusion, 42, 4-9), employed world wide, (Anderlini P et al., 1997, Transfusion, 37, 507-512; Kang, E M., 2002, Blood, 99, 850-855), and CD34+ cells were collected by standard leukapheresis techniques and the CD34+ stem cells, (healthy first cell type), isolated by standard magnetic bead technologies, Dyral). The cells were frozen until use and RNA extracted by standard methods. The RNA was appropriately converted, labelled, hybridized to microarray platforms and RNA expression levels inferred by appropriate laser scanning or by radioactive detection-methods. Bioinformatic analysis of the resulting data provided snapshots of relative gene activity across the genome, with the Affymetrix platform, U133A having in excess of 20,000 well annotated human genes, (or parts of genes), on the platform.

Microarray Analysis of Human CD34+ Hemopoietic Stem Cells Using the Atlas Plastic 8K Array Protocol

Purification of CD34+ Hemopoietic Stem Cells from a 49 Year Old Male Patient

Purification of CD34+ Cells from Whole Blood

Samples were obtained from a patient undergoing leukapheresis at the Royal North Shore Hospital, Sydney. Samples were obtained with prior Ethics Committee approval. White blood cells were concentrated using Ficoll Paque plus (Amersham Biosciences #17-1440-03; Piscataway N.J.) according to the manufacturers instructions. CD34+ cells were isolated from the white cell population CD34 Progenitor Cell selection system (Dynal #113.01) respectively according to the manufactures instructions.

Atlas RNA Extraction Procedure

I. 1 ml of Trizol added directly to the magnetic bead/cell complex and samples stored at −70° C.
II. Samples removed from −70° C. and thawed on ice.
III. Samples mixed well and left at room temperature for 5 minutes to dissociate nucleoprotein complexes.
IV. 0.5 ml of beads/cell removed into a clean RNase free 1.5 ml centrifuge tube and the tube placed on a magnetic seperator for 60 seconds and the bead free supernatant removed into a clean tube.
V. The samples were then spun @ 12,000×g for 10 minutes @ 4° C. to remove high molecular weight DNA and other contaminants.
VI. The supernatant removed into a clean tube and 100 μl of 100% chloroform added and the samples mixed vigorously by hand for 15 seconds then incubated @ room temperature for 2-3 minutes.
VII. The samples were then spun @ 12,000×g for 10 minutes @ 4° C. to separate the phases.
VIII. The upper aqueous phase was removed into a clean tube ensuring the pipette tip stayed away from the interface and 1 μl of 20 mg/ml glycogen added and the samples vortexed.
IX. An equal volume of 100% (0.25 ml) was added the tubes vortexed then left @ room temp for 10 minutes.
X. The samples were then spun @ 12,000×g for 10 minutes @ 4° C. to pellet the RNA.
XI. The supernatant removed and the pellet washed with 0.75 ml of 80% ethanol to removed inhibitors of the cDNA synthesis reaction, vortexed briefly then spun @ 7,500×g for 5 minutes @ 4° C. to pellet the RNA.
XII. Step 11 was repeated a further X1.
XIII. The pellet was then spun in a microfuge for 10 seconds the residual ethanol removed and the pellet immediately resuspended in 25 μl of RNase free water. NB if the pellet dries out then it is very difficult to resuspend the RNA and the 260/280 ratio will be less than 1.6.
XIV. The OD 260/280/310 is then recorded.
   cDNA Synthesis

I. The following reagents were prepared for each cDNA synthesis reaction in thin-wall 0.5 ml RNase free tubes.

| | μl added | | |
|---|---|---|---|
| | CD34+ cells | T-cells | Control RNA |
| RNA | 3.5 (200 ng) | 1 (243 ng) | 1 (1000 ng) |
| cDNA Primer (10 μM) | 1 | 1 | 1 |
| SMART Oligo (10 μM) | 1 | 1 | 1 |
| Deionised water | | 2.5 | 2.5 |

II. The contents were mixed and spun briefly in a microfuge.
III. The samples were incubated @ 70° C. for 8 minutes to denature the RNA.

IV. While the RNA was being denatured the following master mix was prepared:

μl added

| | Per reaction | 3.5 reaction |
|---|---|---|
| 5× first strand buffer | 2 | 7 |
| DTT (20 mM) | 1 | 3.5 |
| 50× dNTP mix | 1 | 3.5 |
| Total volume | 4 μl | 14 μl |

V. Tubes were removed from the PCR machine and cooled on ice for 2 minutes then spun briefly to collect contents.
VI. Samples were then incubated @ 42° C. for 2 minutes.
VII. 0.5 μl of Powerscript Reverse Transcriptase was added per reaction (1.75 μl) and the master mix mixed well by pipetting.
VIII. 4.5 μl of the complete master mix was added to each sample and control tube and the samples then incubated @ 42° C. for 60 minutes then the samples transferred to ice.
IX. 40 μl of 10 mM Tris 1 mM EDTA pH 7.6 was added to each sample.
X. The tubes were heated 72° C. for 7 minutes then stored @ −70° C. until required.
   Atlas CDNA Amplification by Long Distance PCR

NB the number of cycles has to be determined for each sample to ensure that the reaction does not plateau. Amplification reactions were set up in triplicate for each sample (2 test and 1 control) and in duplicate for the control placenta (1 test and 1 control).
I. The thermal cycler was preheated to 95° C.
II. In a 0.5 ml thin walled tube, 5 μl of First-Strand cDNA was mixed with 37 μl of deionised water.

III. The following master mix was prepared:

| | Per rxn | 8.5 rxn |
|---|---|---|
| 10× PCR buffer | 5 μl | 42.5 μl |
| 50× dNTP mix (10 mM) | 1 μl | 8.5 μl |
| PCR primer (10 μM) | 1 μl | 8.5 μl |
| Advantage Polymerase | 1 μl | 8.5 μl |
| Total volume PCR mix | 8 μl | 68 μl |

IV. Master mix vortexed then spun briefly in a microfuge.
V. 8 μl of master mix was added to the appropriate tubes and the sample mixed well by pipetting.
VI. Tubes bverlayed with 55 μl of mineral oil.

VII. Thermal cycling commenced as follows:

Hybaid PCR machine

| | | | |
|---|---|---|---|
| x cycles | 95° C. | 1 minute | |
| | 95° C. | 15 sec | |
| | | 65° C. | 30 sec |
| | | 68° C. | 3 minutes. |

VIII. All tubes subjected to 15 cycles of PCR as above, then the test samples removes (i.e ×2 CD34+/×2 T-cell/×1 control) and these tubes placed on ice.
IX. 10 μl of each control sample was removed into a clean tube and the remainder of the mix subjected to a further 3 cycles of PCR as above.
X. Step 9 was repeated a further ×2 to give a final of 24 cycles of PCR.
XI. These samples were the analysed on a 1.2% agarose gel using the 1 kb DNA size markers.
   Atlas Spin Column Purification of PCR Products
   NB Using the Nucleospin System NOT the Chroma Spin System.
I. 28 ml of 95% ethanol added to buffer NT3.
II. Volume of PCr products adjusted to 100 μl with 50μl of TE buffer pH 7.5 and the samples mixed well by pipetting.
III. 400 μl of NT2 buffer added to the sample and the sample mixed well.
IV. Nucleospin columns placed in 2 ml collection tubes and the sample pipetted onto the filter. The samples were then centrifuged @ 14,000 rpm for 1 minute and the collection tube discarded.
V. Column placed into a fresh collection tube and 500 μl of buffer NT3 added. The samples were then centrifuged @ 14,000 rpm for 1 minute and the collection tube discarded.
VI. Step 5 was repeated a further ×2.
VII. Column placed into a fresh collection tube and the samples were then centrifuged @ 14,000 rpm for 1 minute to remove residual ethanol.
VIII. Column placed into a clean 1.5 ml centrifuge tube and 50 μl of buffer NE added DIRECTLY onto the filter (NB make sure the pipette DOES NOT touch the filter surface).
IX. The sample was allowed to soak for 2 minutes then centrifuged @ 14,000 rpm for 1 minute to elute the purified PCR fragments.
X. Determine the OD260/280/310.
   Atlas Spin Column Purification of the T-Cell PCR Product #2
   NB Purification Repeated to Increase PCR Product Yield.
I. Volume of PCR products adjusted to 100 μl with 50 μl of TE buffer pH 7.5 and the samples mixed well by pipetting.
II. 400 μl of NT2 buffer added to the sample and the sample mixed well.
III. Nucleospin columns placed in 2 ml collection tubes and the sample pipetted onto the filter. The samples were then centrifuged @ 14,000 rpm for 1 minute and the collection tube discarded.
IV. Column placed into a fresh collection tube and 500 μl of buffer NT3 added. The samples were then centrifuged @ 14,000 rpm for 1 minute and the collection tube discarded.
V. Step IV was repeated a further ×2.
VI. Column placed into a fresh collection tube and the samples were then centrifuged @ 14,000 rpm for 1 minute to remove residual ethanol.
VII. Column placed into a clean 1.5 ml centrifuge tube and 50 μl of buffer NE added directly onto the filter (NB make sure the pipette does not touch the filter surface).
VIII. The sample was allowed to soak for 2 minutes then centrifuged @ 14,000 rpm for 1 minute to elute the purified PCR fragments.
IX. Determine the OD260/280/310.
   Atlas SMART cDNA Probe Labelling
I. Need to label 500 ng of each sample to produce the appropriate probes. NB the final specific activity of the probes should be 10⁷.
II. Preheated a thermal cycler to 97° C.

III. In separate 0.5 ml thin walled tubes 1 μl of Random primer mix was added along with appropriate volume of purified PCR products and deionised water (see below).

| | CD34+ | T-cell | Control |
|---|---|---|---|
| Purified PCR product | 23 μl | 18.9 μl | 11.9 μl |
| Deionised water | 10 μl | 14.4 μl | 21.1 μl |

IV. Samples denatured for 8 minutes @ 97° C.

V. While samples were denaturing the following master mix was prepared:

| | CD34+/T-cells | | Control |
|---|---|---|---|
| 10× Label Buffer | 11 μl | 10× Label Buffer | 5.5 μl |
| 10× dNTP mix for dA | 11 μl | 10× dNTP mix for dA | 5.5 μl |
| ³³P-dATP | 11 μl | ³³P-dATP | 5.5 μl |
| Total volume mix | 33 μl | | 16.5 μl |

VI. After denaturation samples were mixed well by pipetting and tubes briefly spun.
VII. Thermal cycler temperature dropped to 50° C., tubes incubated @ 50° C. for 3 minutes.
VIII. 1 μl of Klenow enzyme was added per reaction to the master mix and mixed well by pipetting.
IX. After incubation 16 μl of master mix was added to the appropriate tube, the contents mixed and the samples immediately returned to the thermal cycler.
X. Tubes were incubated @ 50° C. for 30 minutes and the reaction stopped by the addition of 2 μl of 0.5M EDTA.
   Atlas Spin Column Purification of the ³³P-dATP PCR Products
I. NT2 buffer added to a final volume of 400 p (350 μl of buffer) to the sample and the sample mixed well.
II. Nucleospin columns placed in 2 ml collection tubes and the sample pipetted onto the filter. The samples were then centrifuged @ 14,000 rpm for 1 minute and the collection tube discarded.
III. Column placed into a fresh collection tube and 350 μl of buffer NT3 added, The samples were then centrifuged @ 14,000 rpm for 1 minute and the collection tube discarded.
IV. Step 3 was repeated a further ×2.
V. Column placed into a clean 1.5 ml centrifuge tube and 100 μl of buffer NE added directly onto the filter (NB make sure the pipette does not touch the filter surface).
VI. The sample was allowed to soak for 2 minutes then centrifuged @ 14,000 rpm for 1 minute to elute the purified PCR fragments.
VII. Determine the CPM of the purified probes.
VIII. 2 μl of purified material added to 5 ml of scintillant and the CPM recorded on the ³²P chanel.
   Atlas Srray Hybridisation
I. Hybridisation bottles filled 80% full with deionised water and heated to 60° C. At the same time 2×25 ml of plastichyb warmed to 60° C. in separate 50 ml falcon tubes.
II. The arrays were placed in the pre-warmed water with the printed side (non-shiny) facing inwards.
III. 10 ml of plastichyb (×2) was placed in a fresh container. 50 μl of 20×SSC was combined with 50 μl of smart blocking reagent and heated @ 95° C. for 5 minutes then snap chilled on ice for 2 minutes. This solution was then combined with the plastichyb.
IV. The water was drained off the array and the plastichyb added and the arrays prehybridised for 30 minutes @ 60° C.
V. 50 μl of 20×SSC was combined with 50 μl of smart blocking reagent and added to the purified probes. The probes were boiled for 10 minutes and chilled on ice for 2 minutes.
VI. The above solutions were combined with the remaining 2×15 ml of plastichyb, the solutions mixed thoroughly, the prehyb discarded and the hybridisation solution added to the appropriate bottles.
VII. The arrays were hybridised overnight @ 60° C., making sure that they were level at all times.
   Atlas Washing and Exposure
I. The hybridisation solution was discarded into 50 ml falcon tubes and the hybridisation bottles filled 80% with pre-warmed (60° C.) high salt buffer (2×SSC/0.1×SDS) and the sample incubated @ 58° C. for 5 minutes to remove residual radiation.
II. The wash buffer was removed and step I repeated.
III. The bottle was then filled 80% with low salt wash buffer (0.1×SSC/0.1% SDS) and the array incubated @ 58° C. for 5 minutes.
IV. Step III was repeated a further ×1.
V. The temperature of the hybridisation oven was reduced to 30° C. and the bottles again filled 80% with room-temperature low salt buffer and incubated for a further 5 minutes.
VI. The arrays were then removed from the hybridisation bottles into 500 ml beakers. filled with low salt wash buffer.
VII. The arrays were rinsed by dipping in the beaker several times then finally removed very slowly ensuring no large droplets adhered to the array surface.
VIII: The array were air dried to completion, taped inside a phosphor cassette and the screen placed in direct contact with the array.
   The arrays were exposed at 1 day and 7 days.
   Affymetrix Microarray Analysis of Stem Cells from Individuals of Various Ages Gene-Expression Analysis with Affymetrix Genechip® Arrays

The data obtained to support the present invention include results from gene expression analysis on an independent microarray platform. Gene expression analysis was performed with Affymetrix® GeneChip® technology. For detailed information, refer to the Affymetrix web page (www.affymetrix.com).

All the steps of the process—RNA isolation and purification, cRNA synthesis and fragmentation, hybridization, staining, scanning of the array—have been carried out with equipment and protocols recommended by Affymetrix. The software used for data analysis is Microarray Suite version 5.0 (Affymetrix®)). Detailed information about the statistical algorithm used can be found in the Affymetrix web page (www.affymetrix.com).

Affymetrix Sample Processing

Gene-expression analysis was performed starting from the cell samples included in RNAlater. The internal processing code for these samples are shown in Table 1 below.

TABLE 1

| Sample code | Code # |
|---|---|
| R1 Male aged 22 | SE-488 |
| R2 male aged 23 | SE-489 |

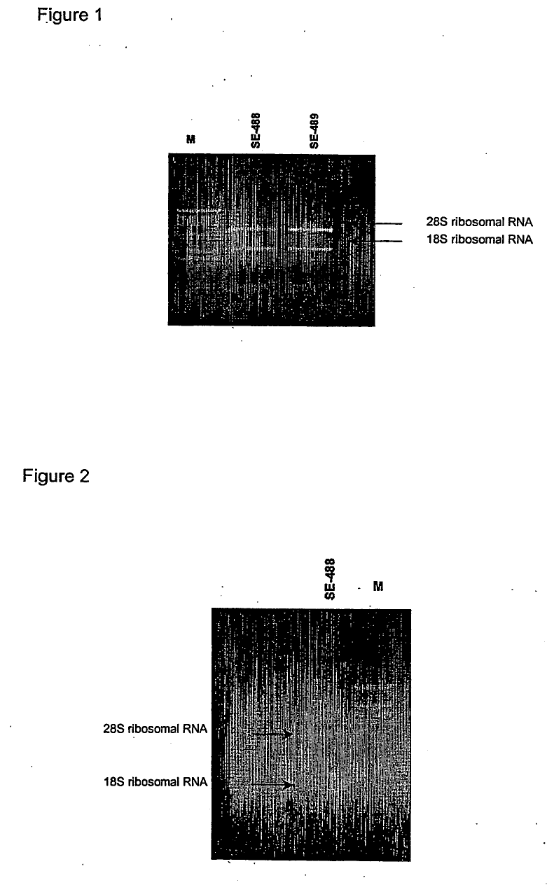

Total RNA was isolated with TRIzol Reagent (Life Technologies) by following the manufacturer's instructions. Each sample was resuspended in 22 μl of RNase free water. A fraction was analyzed by agarose gel electrophoresis (FIG. 1).

Both samples were mixed before purification and the mix was called SE488. Purification was performed with the RNeasy kit (Q uiagen). RNA was eluted in 35 μl of RNase free water, and concentrated on a speed-vac to a final volume of 12 μl. The quantity and quality of the purified RNA were checked by spectophotometry and agarose gel electrophoresis (FIG. 2).

Affymetrix Synthesis of Biotin-Labeled cRNA

Starting from purified total RNA, cDNA was synthesized with SuperScript Choice System Kit (Life Technologies) by following the Affymetrix Expression Analysis Technical Manual protocol.

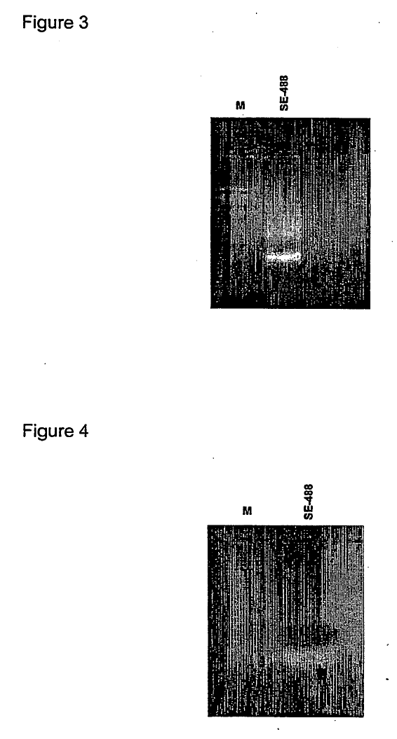

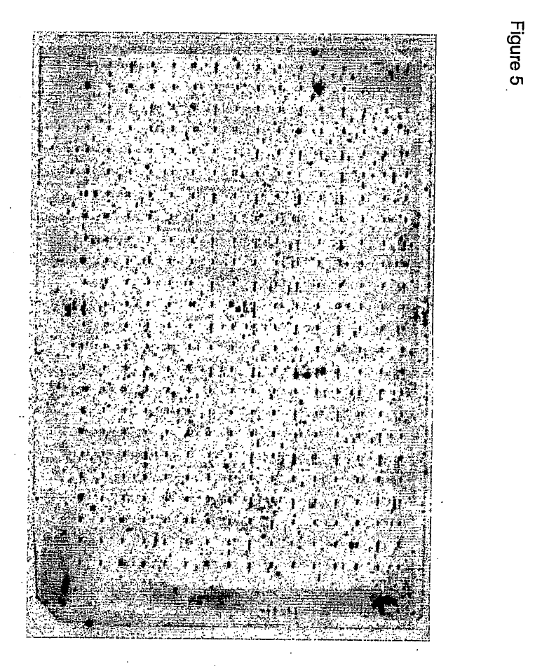

cRNA was synthesized from cDNA by following the Enzo BioArray HighYield RNA Transcript Labeling (T7) kit. cRNA was purified with the Affymetrix GeneChip Sample Cleanup Moduke Kit and eluted in 22 μl of RNase-free water (FIG. 3 and Table 2).

Concentration and purity of purified cRNA. High purity RNA should have an OD value of>1.9. Thus the RNA from the pooled samples was of sufficient purity for further processing.

TABLE 2

| Sample | Concentration (μg/μl) | Purity (280 nm) |
|---|---|---|
| SE488 | 2.13 | 2.17 |

Purified cRNA was fragmented before hybridization on the array (FIG. 4).

Hybridization and Scanning of Affymetrix U133A Array—Analysis of Controls

There are two fundamental parameters for evaluating the quality of the hybridization mixes: presence of spike controls, and 3′/5′ ratio of housekeeping genes.

Spike controls are control oligonucleotides included in the hybridization mix. The detection of these oligonucleotides upon scanning indicates that the hybridization, washing, staining and scanning steps were properly performed. The spike controls used are BioB, BioC, BioD and Cre. BioB is the spike control with the lowest number of molecules in the mix, which makes it the preferred control for evaluating the sensitivity of the assay

Housekeeping controls are probes for genes constitutively expressed in all tissues and under all circumstances. The probes on the array are designed to hybridize to the 3′, middle and 5′ areas of the housekeeping genes. The 3′/5′ ratio is an indicator of the integrity of the synthesized cRNA, which in turn reflects the integrity of the original RNA. Thus, a 3′/5′ ratio of 1 means total integrity of the starting RNA and the synthesized cRNA. This ratio varies depending on the tissue of origin and the RNA handling conditions. Affymetrix recommends using cRNA preps with a 3′/5′ ratio<3.

The constitutive expression of housekeeping genes varies from tissue to tissue and as a consequence of experimental conditions, as reflected in the literature. Affymetrix strongly recommends GAPDH as a true housekeeping gene.

TABLE 3
GAPDH 3′/5′ ratio and BioB spike
control values from sample SE488.

| | | housekeeping |
|---|---|---|
| sample code | spike control BioB | GAPDH (3′/5′) |
| SE488 (HGU133A) | P | 1.38 |
| SE488 (HGU133B) | P | 1.23 |

Representative genes were selected for methylation analysis. It should be noted that currently no high throughput technology exists to directly measure the genome-wide methylation status of the entire human genome, (this methodology is in its infancy; Adorjan et al., 2002, Nucleic Acids Research, 30, e21; Yan P S et al., 2000, Clinical Cancer Research, 6, 1432-1438); Gitan R S et al., 2001, Genome Research, 12, 158-164), and it was for this reason that we applied an initial filter to the aging genome on the basis of RNA expression profiling.

Combined Data Analysis From the Atlas and Affymetrix Gene Expression Profiling

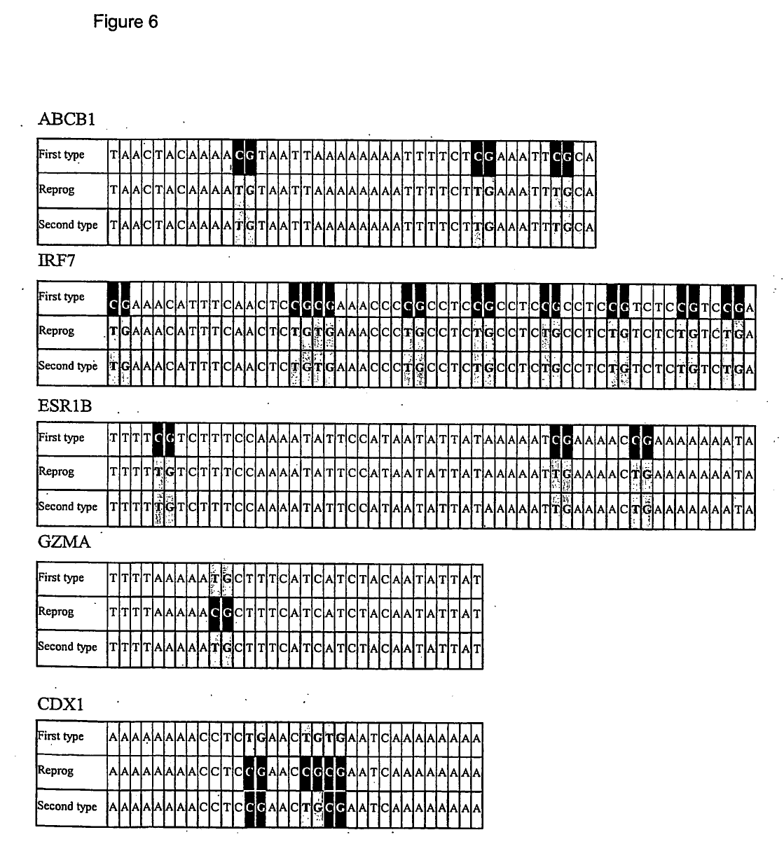

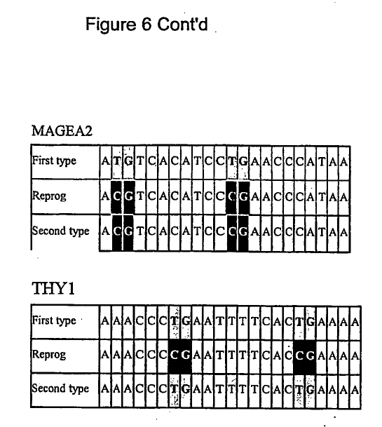

Example of genes upregulated and downregulated in aging cells as determined by microarray analysis using both Affymetrix and Atlas platforms. These expression profiles give a genome-wide analysis of activity in young and old stem cell populations thus yielding an idea of the important genes in the aging process. Such genes are then used in methylation analysis to confirm the optimal reprogramming of aged cells back into a younger phenotype (see FIG. 6 for details of how such analyses were performed).

Some genes whose activities have altered significantly during the aging process are shown in Table 4.

An example of genes upregulated and downregulated in aging cells as determined by microarray analysis using both Affymetrix and Atlas platforms are shown in Table 4. These expression profiles provide a genome wide analysis of mRNA activity in young and old stem cell populations and yield a measure of the important genes that alter during the aging process. Such gene regions were then selected for DNA methylation analyses and were subsequently used to confirm the optimal reprogramming of aged cells back into a younger phenotype.

TABLE 4

| Down-regulated genes | Up-regulated genes |
|---|---|
| RPS11 | HLA-DRB4 |
| Vimentin | HSPA1B |
| Cofilin | HSPA1A |
| RPL38 | MAPK3 |
| L3MBTL | CDPEB |
| CACNA1E | |

Results
Reprogramming

It will be appreciated by those knowledgeable in the art that the treatment or reprogramming and subsequent transplantation of certain adult stem cells requires meeting strict ethical guidelines and the satisfactory completion of extensive documentation in various countries. In addition, a technical block is that adult hematopoietic stem cells undergo very little cell division ex vivo. Stem cells need to be transplanted in vivo to the same individual, where they home to the bone marrow and undergo cell division. Hence providing proof of principle that the reprogrammed DNA methylation state of an adult human stem cell is stably inherited over a number of cell divisions in vivo, has a number of currently insurmountable hurdles stemming from ethical and technical considerations. We have therefore provided proof of principle of stable inherited methylation changes in a different human system using cells in which the methylation state of a fibroblast cell is reprogrammed to that of an immune system T cell.

Selecting Genes Altered During the Aging Process

To determine which genes have their DNA methylation signatures altered during the aging process, we have set up an initial molecular filter based on altered changes in expression profiling, (RNA expression levels of genes). Since, in general, increased methylation leads to a shutting down of gene activity, whereas demethylation leads to an increase in gene activity, we have sought to find those genes whose activity status alters during the aging process. To those knowledgeable in the art, we have used adult CD34+ stem cells from mobilized peripheral blood from individuals of different ages, varying from their early 20s to their late 60s, (de la Rubia, J., et al., 2002, Transfusion 42, 4-9). We have utilized commercial Affymetrix microarrays, (www.affvmetrix.com), as well as other commercial platforms, Atlas, for measuring altered RNA expression profiles between young and old individuals. We have selected for genes whose expression levels have either been downregulated or upregulated during the aging process. Following such a selection, the methylation status of appropriate regulatory or gene controlling regions can be examined.

First Cell Type (Fibroblast Cells) and Second Cell Type (T-Cells)

Take fibroblast cells with a particular DNA methylation signature genome wide and expose the cells for a period of hours to an extract derived from T cells of a cell line, or T cells from the peripheral blood of a healthy individual. Transfer the cells to fresh medium and allow the cells to undergo many cell divisions. Monitor the changing phenotypic status of the reprogrammed cells whose molecular decorations change from those of the fibroblast cell to those of a T cell, (cell surface molecules provide one handle on the phenotypic status). Isolate the Agenomic DNA from the reprogrammed cells and determine the methylation signature of the genome, comparing it to that of the first cell type and second cell type genomes.

Blood Derived Stem Cells

A similar protocol would be applied in the case of stem cell reprogramming, following appropriate Ethical Approval.

Take blood-derived bone marrow stem cells from an aged first cell type or a first cell type which has been treated with DNA damaging agents. These stem cells have been shown to have a corrupted methyl cytosine (MC) signature in their genomes. Expose the cells for a period (of a few minutes to many hours) to a cell-free extract obtained from a sample of stem cells from a young healthy second cell type then transfer the exposed cells to fresh culture medium and incubate the cells for a period before transferring the treated stem cells back into the host with the damaged stem cells.

First cell type represents a cell type prior to any reprogramming treatment. Second cell type represents the destination towards which the first cell type needs to be moved. “Reprogrammed” denotes the cells that have been treated with an lysate, extract or compound from the second cell type and which are now somewhere along a trajectory from the characteristics of the first cell type to the second cell type.

Cell types. There are at least 300 cell types in a human being from fertilization onwards and these have classically been defined on the basis of their morphology under the light microscope. Thus the members of the hematopoietic lineage, such as basophils, neutrophils, eosinophils, megakaryocytes and platelets are easily distinguishable from each other and from endothelial cells, skin cells, and cells of the nervous system such as neurons and glial cells. The molecular taxonomy of cell types has revealed that antibodies recognizing cell surface proteins and/or internal proteins of a cell, allow finer distinctions to be made between cell types. Gene expression profiling using microarray-type technologies, (which measure the abundance and type of mRNA molecules within a cell), can also be used for making distinctions between cell types.

Reprogramming from one cell type to another. Cell types are interconvertible using appropriate methodologies, (a first cell type can be reprogrammed to a second cell type). What is unclear in all of these reprogrammings in the prior art is whether the available cell markers are sufficiently robust and numerous enough to reveal whether the reprogrammed cell has completely moved from the first cell type to a second cell type, whether it is only partly reprogrammed, whether the reprogramming is stable, or whether the methods used for the reprogramming have introduced unwanted changes that compromise the ability of the reprogrammed cell to function normally in its new environment. Exact knowledge about reprogrammed cells is particularly critical in the case of stem cell reprogramming, as stem cells are the progenitors of many subsequent cell types. In addition, the markers that are used in the prior art for delineating a cell type, such as an mRNA or a protein, may only be transiently expressed. In contrast, the present inventors have found that methylation signatures are stable indicators of change, certainly in the time frames that are being considered.

Methylation characteristics of the genomes of different cell types. The present invention deals with what we have termed ‘cellular trajectories’. These trajectories lead from one cell type to another (first cell type to a second cell type) and measure how far along a trajectory a reprogrammed cell population lies. The present inventors use the methylation signature from genomic regions of the cell being reprogrammed to determine the position of the reprogrammed cell on the trajectory. For example, a stretch of DNA of 1000 nucleotides in the human genome will have a number of cytosines, each of which can exist in either a methylated (M) or unmethylated (u) state. This run of cytosines in the first cell type may be M M M M M, whereas in the second cell type it may be u M u M u. These characteristic signatures are unique and stable for each cell type, so one can determine whether a cell which is being reprogrammed from a first cell type to a second cell type, has actually made it as far as the desired second cell type, or whether it is only partly along the trajectory. Thus, if the reprogrammed cell has a methylation signature of u M u M u then it has reached the destination of the second cell type. If, however, the treated cell is u M M M u then it is only partly along the trajectory. However, if it is u u u M u then there is a problem, since the second position has attained a novel state that is not present in either the first cell type or second cell type.

Levels of Signatures. The genomic methylation signatures that are chosen to reveal where along a trajectory a reprogrammed cell is located at any particular time, can be described at many levels. For example, a contiguous stretch of DNA that is in either a coding or a non-coding region of the genome can be used. Alternatively, stretches of DNA from different parts of the genome, say one stretch from each of the 23 pairs of chromosomes, or multiple stretches from a single chromosome can be used. We illustrate the data of the present invention by using stretches of DNA that are in the vicinity of 7 genes, (termed ABCB1, IRF7, ESRIB, GZMA, CDX1, MAGEA2,and THY1) and only those cytosines that are adjacent to guanines, that is CG doublets are used.

DNA methylation signature is superior to any other indicator of the distance along a cellular trajectory. The prior art is imprecise in defining molecular cell types, since if antibodies are used against cell surface proteins, it may be found, for example, that the T cell receptor complex is present on the surface of a cell, but insufficient other protein classes are present to classify that cell as a functional T cell of the immune system. Similarly with mRNA expression profiling, genes can be expressed in different cell types at various levels, and determining whether the level of expression is sufficiently high to delineate that cell as of a particular type is extremely difficult. In addition, and most importantly, mRNA and protein profiles can be transient, since each is subjected to degradative enzymes, sequestration in multi-subunit complexes within the cell and each mRNA transcript and proptein has different half lives in different genetic backgrounds. In contrast, the present inventors have found that methylation signatures are stably inherited over cell divisions.

Measuring DNA methylation signatures overcomes problems of assigning cell type identity and is powerful in revealing inappropriate alterations that arise from reprogramming. First, DNA methylation itself is an indicator of what is active or inactive within a particular cell type. Hence in moving from a first cell type to a second cell type, a methylation signature in a specially selected genomic region, (such as a promoter or regulatory region of a gene), will indicate whether a gene has been inappropriately silenced in a treated or reprogrammed cell. If it has been silenced, no mRNA or protein product will be made from that locus. Measuring whether that mRNA or protein product is present in a cell by conventional methods is very difficult, particularly if the mRNA or protein is of low abundance. There are no such problems with DNA methylation signatures since they are essentially binary: either a particular cytosine is methylated, or it is not. In addition, if the methylation signature is novel, and belongs neither to the first cell type or second cell type, then the treated or reprogrammed cell type will be deemed inappropriate for clinical use, for example.

Experimental

DNA sequences were selected from seven regions of the human genome. The methylation status of the regions was determined by DNA sequencing after bisulphite modification of the DNA. The first cell type represents the cell type prior to any reprogramming treatment. The second cell type represents the destination towards which the first cell type needs to be moved. “Reprogrammed” denotes the cells that have been treated with an extract from the second cell type and the methylation status of their DNA sequences; the signature is indicative of the changes that have occurred at the DNA level as a result of the reprogramming treatment.

FIG. 6 shows that by analyzing DNA methylation signatures from cells that have been reprogrammed, a determination can be made of the extent to which reprogrammed cells have been moved towards the desired second cell type state. By comparing the methylation signature of the reprogrammed cell with that of the methylation signature of the untreated first cell type as well as of the desired end point second cell type, it can be determined how far the reprogrammed cell has been pushed down the trajectory from the first cell type to that of the desired second cell type. As DNA methylation profiles are stably reproduced during cell division, the reprogrammed cell will inherit and subsequently faithfully reproduce its new reprogrammed signature. If the methylation signature of the reprogrammed cell is different to the desired end point of the second cell type, then the reprogramming is incomplete and the reprogrammed cell may not act in the same way as the desired end point cell type. Therefore, the reprogrammed cell would not be optimal for transplantation purposes or other desired uses. Only when the reprogrammed cell has adopted the methylation profile of the end point second cell type can that reprogrammed cell be utilised for transplantation or other purposes.

The figure shows that by analyzing methylation signatures from cells that have been treated or reprogrammed, a determination can be made of the extent to which treated or reprogrammed cells have been moved towards the desired cell type state. By comparing the methylation signature of the treated or reprogrammed cell with that of the methylation signature of the untreated first cell type as well as of the desired end point second cell type it can be determine how far the treated or reprogrammed cell has been pushed down the trajectory from the first cell type to that of the desired second cell type. As methylation profiles are stably reproduced during cell division, the treated or reprogrammed cell will inherit and subsequently faithfully reproduce its new reprogrammed methylation signature. If the methylation signature of the treated reprogrammed cell is different to the desired end point of second cell type, then the treatment or reprogramming was incomplete and the treated or reprogrammed cell may not act in the same way as the desired end point second cell type. Therefore, the treated or reprogrammed cell would not be optimal for transplantation purposes, for example. Only when the treated or reprogrammed cell has adopted the methylation signature or profile of the second cell type or any other desired cell type can that reprogrammed cell be utilised for transplantation purposes.

From the above results it can be clearly seen that after the treatment or reprogramming of the first cell type, four of the genes tested (ABCB1, IRF7, ESR1 B and MAGEA2) were faithfully reprogrammed at the methylation level and one gene, CDX1, showed partial epigenetic reprogramming. However, two genes, THY1 and GZMA-were not successfully reprogrammed. GZMA is a T cell and natural killer cell-specific serine protease, thus the incorrect reprogramming suggests that these cells may not be optimal for transplantation purposes.

The methylation signature of GZMA in the reprogrammed cells is indicative of gene silencing. This would be extremely-difficult to-detect using conventional gene expression profiling or proteomic studies as the gene product is no longer present. The methylation results however are an unambiguous indicator of any potential problems in the reprogrammed cell.

This approach demonstrates the potential of methylation analysis to correctly discriminate between reprogrammed cell types, and allows a powerful means to select optimal cells for transplantation methodologies. This technique is also ideal to optimise cell reprogramming protocols independent of transplantation.

From the results in FIG. 6 it can be clearly seen that after reprogramming the first cell type, four of the regions tested (ABCB1, IRF7, ESR1 B and MAGEA2) were faithfully reprogrammed at the methylation level while one region, CDX1, showed partial reprogramming. However, two genes, THY1 and GZMA were not successfully reprogrammed. GZMA is a T cell and natural killer cell-specific serine protease, thus the incorrect reprogramming suggests that these cells may not be optimal for transplantation purposes.

Our approach demonstrates the potential of DNA methylation analysis to correctly discriminate between reprogrammed cell types, and provides a powerful means of selecting optimal cells for transplantation methodologies. This technique is also ideal for optimizing cell reprogramming protocols independent of transplantation.

Methylation signature changes within cell types. The power of the present methylation signature protocol can be realized when it comes to analysing cell populations that are of the same cell type, and where transcriptomic and proteomic analyses are, very difficult. A preferred case is reprogramming old hematopoietic stem cells to younger stem cells and old T cells of the immune system to younger cells of the immune system. In these cases, the changes at the mRNA and protein levels are expected to be subtle and conventional technologies are stretched. Methylation signatures using chosen loci from the human genome are able to detect whether the reprogramming of old to young has been successful, and most importantly, whether inappropriate changes have been avoided.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-21. (canceled)

22. A method for altering a characteristic or state of a cell comprising:
treating a first cell type with an agent capable of altering a characteristic or state in a cell, wherein the agent is an extract, lysate, cellular component or mixture thereof derived or obtained from a second cell type having a desired characteristic or state; and
determining the degree of alteration in the treated cell by measuring a methylation signature within the genome of the treated cell, wherein a given methylation signature is indicative of an altered characteristic or state of the treated cell.

23. The method according to claim 22 wherein the first cell type is selected from the group consisting of a cell derived from an individual suffering from age-related disabilities, a disease such as cancer, an autoimmune disease, cardiovascular problems such as myocardial infarction or ischemia, stem cell, T cell or monocyte of the immune and hematopoietic system, normal cell, and mixtures thereof.

24. The method according to claim 23 wherein the first cell type is a stem cell.

25. The method according to claim 22 wherein the second cell type is any cell type or combination of cell types.

26. The method according to claim 25 wherein the second cell type is selected from the group consisting of a cell derived from a healthy individual, stem cell, T cell or monocyte of the immune and hematopoietic system, normal cell, and mixtures thereof.

27. The method according claim 25 wherein the first and second cell types are selected from the group consisting of cells of the human haematopoietic system, stem cells, and epithelial cells.

28. The method according claim 27 wherein the second cell type is derived from a normal or healthy individual of a cell type similar to the first cell type.

29. The method according claim 28 wherein the second cell type is a stem cell.

30. The method according to claim 22 wherein the first cell type cell and the second cell type cell are of the same cell type from the same species.

31. The method according to claim 22 wherein the first cell type and the second cell type are not of the same cell type.

32. The method according to claim 22 wherein the first cell type and the second cell type are not of the same species.

33. The method according claim 32 wherein the second cell type is an amphibian cell and the first cell type is human or other mammalian cell.

34. The method according to claim 22 wherein the first cell type is pre-treated so as to make the cell permeable to macromolecules.

35. The method according to claim 34 wherein the cell is pre-treated by electroporation, low temperature thermal shock, or various enzymes such as streptolysin O.

36. The method according to claim 35 wherein the pre-treatment renders the cell temporally permeable.

37. The method according to claim 22 further including:
culturing or growing the treated cell to obtain multiple copies of the treated cell.

38. The method according to claim 37 wherein the treated cell is cultured in any suitable media or host under conditions that are suitable for cell growth and division.

39. The method according to claim 38 wherein the host is a domestic animal selected from the group consisting of bovine, ovine, equine, poultry, and porcine.

40. The method according to claim 22 wherein the methylation signature is a group of cytosines within a region of the genome that has a characteristic methylation signature which corresponds to a specific cell type.

41. The method according claim 22 wherein the methylation signature is determined by the bisulphite modification and subsequent DNA sequence analysis.
